(19) United States
(12) Patent Application Publication
(10) Pub. No.: US 2008/0125764 A1
(43) Pub. Date: May 29, 2008

(54) CRYOPROBE THERMAL CONTROL FOR A CLOSED-LOOP CRYOSURGICAL SYSTEM

(76) Inventors: David W. Vancelette, San Diego, CA (US); Michael V.W. Perkins, Minnetonka, MN (US); Randall C. Lieser, Plymouth, MN (US)

Correspondence Address: AMS RESEARCH CORPORATION, 10700 BREN ROAD WEST, MINNETONKA, MN, 55343, US

(21) Appl. No.: 11/942,059

(22) Filed: Nov. 19, 2007

Related U.S. Application Data
(60) Provisional application No. 60/866,288, filed on Nov. 17, 2006.

(51) Int. Cl. A61B 18/02

(52) U.S. Cl. 606/22; 606/20

(57) ABSTRACT

A cryosurgical system providing for temperature control of individual cryoprobes so as to simplify and increase treatment flexibility in cryoablation procedures. The cryosurgical system provides individual control of multiple cryoprobes in a closed-loop refrigeration circuit. The individual control allows the simultaneous use of multiple cryoprobes in a procedure. Typically six to eight probes are used but additional probes and control thereof is contemplated by this invention. The primary refrigeration circuit's compressor can also be utilized to generate pressurized hot vapor for heating the probe ends. In order to direct the pressurized hot vapor to the probe ends, an internal valving and control system reverses the direction of pressurized gas flow through the cryoprobes, delivering the hot gas immediately to the ends by bypassing the heat exchangers. Thus each cryoprobe can be independently controllable to provide full, partial or no freezing or heating at any time.

### PRIORITY CLAIM

The present application claims priority to U.S. Provisional Application Ser. No. 60/866,288, filed Nov. 17, 2006 and entitled “CRYOPROBE THERMAL CONTROL FOR A CLOSED-LOOP CRYOSURGICAL SYSTEM”, which is herein incorporated by reference in its entirety.

### FIELD OF THE INVENTION

The present invention relates to cryoprobes for use in cryosurgical systems and more specifically to the individual thermal control of multiple cryoprobes for a closed-loop cryosurgical system including the ability to reverse flow for probe heating.

### BACKGROUND OF THE INVENTION

Cryosurgical probes are used to treat a variety of diseases. Cryosurgical probes quickly freeze diseased body tissue, causing the tissue to die after which it will be absorbed by the body, expelled by the body, sloughed off or replaced by scar tissue. Cryothermal treatment can be used to treat prostate cancer and benign prostate disease. Cryosurgery also has gynecological applications. In addition, cryosurgery may be used for the treatment of a number of other diseases and conditions including breast cancer, liver cancer, glaucoma and other eye diseases.

A variety of cryosurgical instruments variously referred to as cryoprobes, cryosurgical probes, cryosurgical ablation devices, cryostats and cryocoolers have been used for cryosurgery. These devices typically use the principle of Joule-Thomson expansion to generate cooling. They take advantage of the fact that most fluids, when rapidly expanded, become extremely cold. In these devices, a high pressure gas mixture is expanded through a nozzle inside a small cylindrical shaft or sheath typically made of steel. The Joule-Thomson expansion cools the steel sheath to a cold temperature very rapidly. The cryosurgical probes then form ice balls which freeze diseased tissue without undue destruction of surrounding healthy tissue.

The use of cryosurgical probes for cryoablation of prostate is described in Onik, Ultrasound-Guided Cryosurgery, Scientific American at 62 (January 1996) and Onik, Cohen, et al., Transrectal Ultrasound-Guided Percutaneous Radial Cryosurgical Ablation Of The Prostate, 72 Cancer 1291 (1993). In this procedure, generally referred to as cryoablation of the prostate, several cryosurgical probes are inserted through the skin in the perineal area (between the scrotum and the anus) which provides the easiest access to the prostate. The probes are pushed into the prostate gland through previously placed cannulas. Placement of the probes within the prostate gland is visualized with an ultrasound imaging probe placed in the rectum. The probes are quickly cooled to temperatures typically below −100° C. The prostate tissue is killed by the freezing, and any tumor or cancer within the prostate is also killed. The body will absorb some of the dead tissue over a period of several weeks. Other necrosed tissue may slough off through the urethra. The urethra, bladder neck sphincter and external sphincter are protected from freezing by a warming catheter placed in the urethra and continuously flushed with warm saline to keep the urethra from freezing.

Rapid re-warming of cryosurgical probes is desired. Cryosurgical probes are warmed to promote rapid thawing of the prostate, and upon thawing the prostate is frozen once again in a second cooling cycle. Moreover, the probes cannot be removed from frozen tissue because the frozen tissue adheres to the probe. Forcible removal of a probe which is frozen to surrounding body tissue leads to extensive trauma. Thus many cryosurgical probes provide mechanisms for warming the cryosurgical probe with gas flow, condensation, electrical heating, etc.

Some devices utilize separate gas types for reheating. Ben-Zion, Fast Changing Heating and Cooling Device and Method, U.S. Pat. No. 5,522,870 (Jun. 4, 1996) applies the general concepts of Joule-Thomson devices to a device which is used first to freeze tissue and then to thaw the tissue with a heating cycle. Nitrogen is supplied to a Joule-Thomson nozzle for the cooling cycle, and helium is supplied to the same Joule-Thomson nozzle for the warming cycle. Preheating of the helium is presented as an essential part of the invention, necessary to provide warming to a sufficiently high temperature.

Various cryocoolers use mass flow warming, flushed backwards through the probe, to warm the probe after a cooling cycle. Lamb, Refrigerated Surgical Probe, U.S. Pat. No. 3,913,581 (Aug. 27, 1968) is one such probe, and includes a supply line for high pressure gas to a Joule-Thomson expansion nozzle and a second supply line for the same gas to be supplied without passing through a Joule-Thomson nozzle, thus warming the catheter with mass flow. Longsworth, Cryoprobe, U.S. Pat. No. 5,452,582 (Sep. 26, 1995) discloses a cryoprobe which uses the typical fin-tube helical coil heat exchanger in the high pressure gas supply line to the Joule-Thomson nozzle. The Longsworth cryoprobe has a second inlet in the probe for a warming fluid, and accomplishes warming with mass flow of gas supplied at about 100 psi. The heat exchanger, capillary tube and second inlet tube appear to be identical to the cryostats previously sold by Carleton Technologies, Inc. of Orchard Park, N.Y.

Still other Joule-Thomson cryocoolers use the mechanism of flow blocking to warm the cryocooler. In these systems, the high pressure flow of gas is stopped by blocking the cryoprobe outlet, leading to the equalization of pressure within the probe and eventual stoppage of the Joule-Thomson effect. Examples of these systems include Wallach, Cryosurgical Apparatus, U.S. Pat. No. 3,696,813 (Oct. 10, 1973). These systems reportedly provide for very slow warming, taking 10-30 seconds to warm sufficiently to release frozen tissue attached to the cold probe. Thomas, et al., Cryosurgical Instrument, U.S. Pat. No. 4,063,560 (Dec. 20, 1977) provides an enhancement to flow blocking, in which the exhaust flow is not only blocked, but is reversed by pressurizing the exhaust line with high pressure cooling gas, leading to mass buildup and condensation within the probe.

Each of the above mentioned cryosurgical probes builds upon prior art which clearly establishes the use of Joule-Thomson cryocoolers, heat exchangers, thermocouples, and other elements of cryocoolers. However, the prior art fails to provide a system in which each probe is independently controlled during a heating and freezing cycle. Furthermore, there remains a need for a cryoprobe that does not require a separate energy source and circuit or separate gas supply and lines for heating so as to minimize and reduce the cost of each probe.

### SUMMARY OF THE INVENTION

The present invention is directed to a system that simplifies and adds more flexibility to cryoablation procedures. As the individual cryoprobes are directed to various treatment areas it is known that a selectable freeze performance would increase system efficiencies as well as provide greater safety to the patient. The present invention provides individual control of multiple cryoprobes in a closed-loop refrigeration circuit. The individual control allows the simultaneous use of multiple cryoprobes in a procedure. Typically six to eight probes are used but additional probes and control thereof is contemplated by this invention. Thus each cryoprobe will be independently controllable to provide full, partial or no freezing at any time.

The present invention allows for individual control of the cryoprobes through switchable valving on the high pressure delivery tubes of the primary refrigerant circuit for each probe. The refrigerant is channeled either through the heat exchangers and to the probe ends or back to the compressor via bypass tubing. Restrictor elements in the bypass tubing are utilized to balance the mass flow in the circuit when rerouting refrigerant out of the probes. A heat exchanger is added to the bypass line for rejecting excess heat in the return refrigerant flow line.

The present invention further provides an energy means for heating the tips of the cryoprobes in a closed-loop cryosurgical system in order to thaw the cryoprobe produced iceballs created during the freezing treatment and/or release the probes from the frozen tissue. In a first embodiment, the present invention provides an alternative to the separate electrical heater element commonly used on cryoprobes in closed-loop cryosurgical procedures. The primary refrigeration circuit's compressor is utilized to generate pressurized hot vapor for heating the probe ends. In order to direct the pressurized hot vapor to the probe ends, an internal valving and control system reverses the direction of pressurized gas flow through the cryoprobes, delivering the hot gas immediately to the ends by bypassing the heat exchangers. Heat control at the tips is controlled by the temperature sensor feedback. Thus the present invention eliminates the need for a separate energy source and circuit system for heating the cryoprobes. The elimination of the heater system further results in smaller diameter and less expensive probes.

The above summary of the various representative embodiments of the invention is not intended to describe each illustrated embodiment or every implementation of the invention. Rather the embodiments are chosen and described so that other skilled in the art may appreciate and understand the principles and practices of the invention. The figures in the detailed description that follows more particularly exemplify these embodiments.

### BRIEF DESCRIPTION OF THE FIGURES

These as well as other objects and advantages of this invention, will be more completely understood and appreciated by referring to the following more detailed description of the presently preferred exemplary embodiments of the invention in conjunction with the accompanying drawings of which:

FIG. 1 is a side view of an embodiment of a cryosurgical system according to the prior art.

FIG. 2 is a schematic view of a heat exchanger system for use in a cryosurgical system of the prior art.

FIG. 3 is a schematic view of a cryosurgical system according to an embodiment of the present invention.

FIG. 4 is a schematic view of a cryosurgical system according to an embodiment of the present invention.

### DETAILED DESCRIPTION

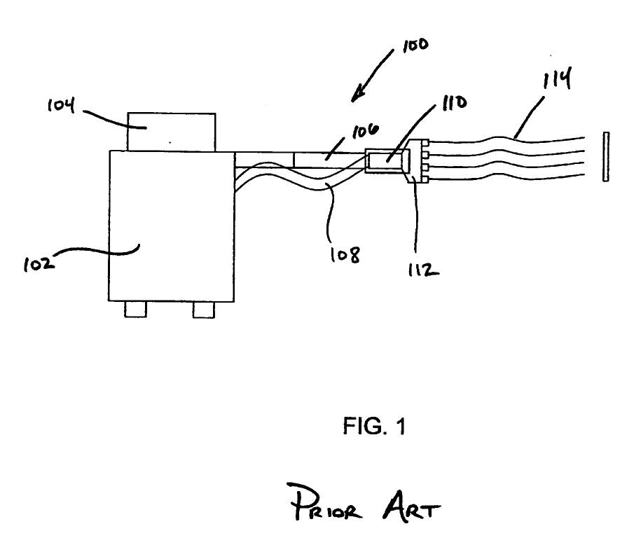

The present invention builds off prior art cryosurgical systems in which a manifold is used to distribute refrigerant to multiple probes. The present invention includes the means to separately heat and cool the individual probes. A prior art closed loop cryosurgical system 100 is illustrated in FIG. 1. Cryosurgical system 100 can include a refrigeration and control console 102 with an attached display 104. Control console 102 can contain a primary compressor to provide a primary pressurized, mixed gas refrigerant to the system and a secondary compressor to provide a secondary pressurized, mixed gas refrigerant to the system. Control console 102 can also include controls that allow for the activation, deactivation, and modification of various system parameters, such as, for example, the flow rates, pressures, and temperatures of the refrigerants. Display 104 can provide the operator the ability to monitor, and in some embodiments adjust, the system to ensure it is performing properly and can provide real-time display as well as recording and historical displays of system parameters. One exemplary console that can be used with an embodiment of the present invention is used as part of the Her Option® Office Cryoablation Therapy available from American Medical Systems of Minnetonka, Minn.

The high pressure primary refrigerant is transferred from control console 102 to a cryostat heat exchanger module 110 through a flexible line 108. The cryostat heat exchanger module 110 transfers the refrigerant into and receives refrigerant out of one or more cryoprobes 114. The particular cryoprobe configuration will depend on the application for which the system is used. For example, a uterine application will typically use a single, rigid cryoprobe, while a prostate application will use a plurality of flexible cryoprobes (which is shown in the embodiment of FIG. 1). If a single, rigid cryoprobe is used, the elements of the cryostat heat exchanger module 110 may be incorporated into a handle of the cryoprobe.

In the prior art, as depicted in FIG. 1, when a plurality of flexible cryoprobes are used, a manifold 112 is connected to cryostat heat exchanger module 110 to distribute the refrigerant among the several cryoprobes. The cryostat heat exchanger module 110 and cryoprobes 114 can also be connected to the control console 102 by way of an articulating arm 106, which may be manually or automatically used to position the cryostat heat exchanger module 110 and cryoprobes 114. Although depicted as having the flexible line 108 as a separate component from the articulating arm 106, cryosurgical system 100 can incorporate the flexible line 108 within the articulating arm 106.

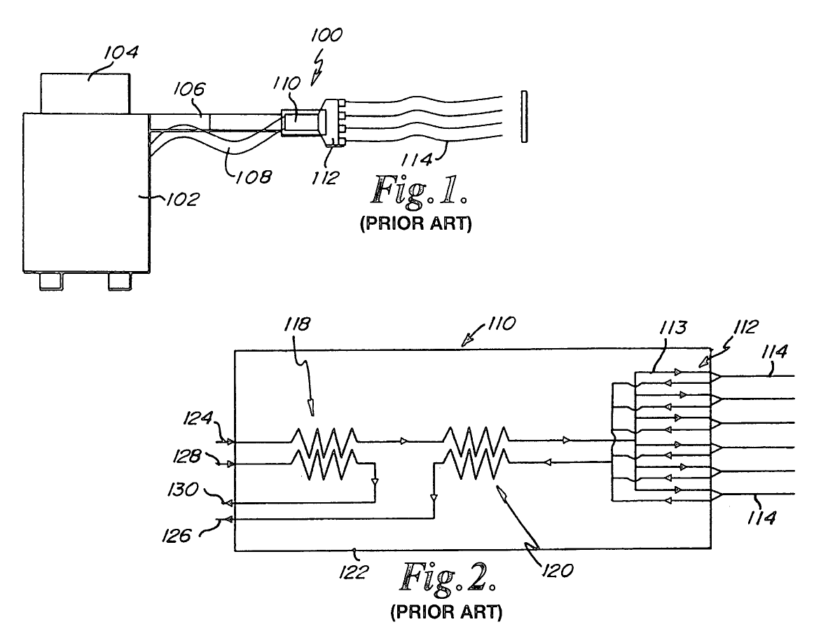

Referring now to FIG. 2, there can be seen a prior art embodiment of a cryostat heat exchanger module 110. The cryostat 110 may contain both a pre-cool heat exchanger, or pre-cooler 118, and a recuperative heat exchanger, or recuperator 120. A vacuum insulated jacket 122 surrounds the cryostat 110 to prevent the ambient air from warming the refrigerant within the cryostat 110 and to prevent the outer surface of the cryostat 110 from becoming excessively cold. High pressure primary refrigerant 124 enters the cryostat 110 and is cooled by high pressure secondary refrigerant 128 that is expanded to a lower temperature in the pre-cool heat exchanger 118. The resulting low pressure secondary refrigerant 130 then returns to the secondary compressor to be repressurized. Since the secondary refrigerant does not flow into the probes 114 (which are brought into direct contact with the patient), a higher pressure can be safely used for the secondary refrigerant 128, 130 than the primary refrigerant 124, 126.

The high pressure primary refrigerant 124 then flows into the recuperator 120 where it is further cooled by the low pressure primary refrigerant 126 returning from the manifold 112. The low pressure primary refrigerant 126 is colder than the high pressure primary refrigerant because it has undergone Joule-Thompson expansion in the plurality of probes 114. Recuperator 120 is preferably incorporated into the cryostat 110. Alternatively, tubing coils inside each probe 114 may act as recuperative heat exchangers in order to reduce insulation requirements and return low pressure refrigerant to the console.

After leaving the recuperator, high pressure primary refrigerant 124 flows into the manifold 112, where it is distributed into multiple flexible probes 114. In one presently contemplated embodiment, six probes are connected to the manifold, but one of skill in the art will recognize that greater or fewer probes may be used depending on the needs of a particular procedure. In each probe 114, the refrigerant 124 flows into a Joule-Thompson expansion element, such as a valve, orifice, or other type of flow constriction, located near the tip of each probe 114, where the refrigerant 124 is expanded isenthalpically to a lower temperature. In one presently preferred embodiment, the Joule-Thompson expansion elements are capillary tubes. The refrigerant then cools a heat transfer element mounted in the wall of the probe, allowing the probe to form ice balls that freeze diseased tissue. The refrigerant then follows low pressure primary refrigerant path 126, exits the manifold 112, travels through the recuperator 120 (where it serves to further cool the high pressure primary refrigerant 124), flows past the precooler 118 and back to the primary compressor in the console, where it is compressed back into high pressure refrigerant 124 so that the above process can be repeated.

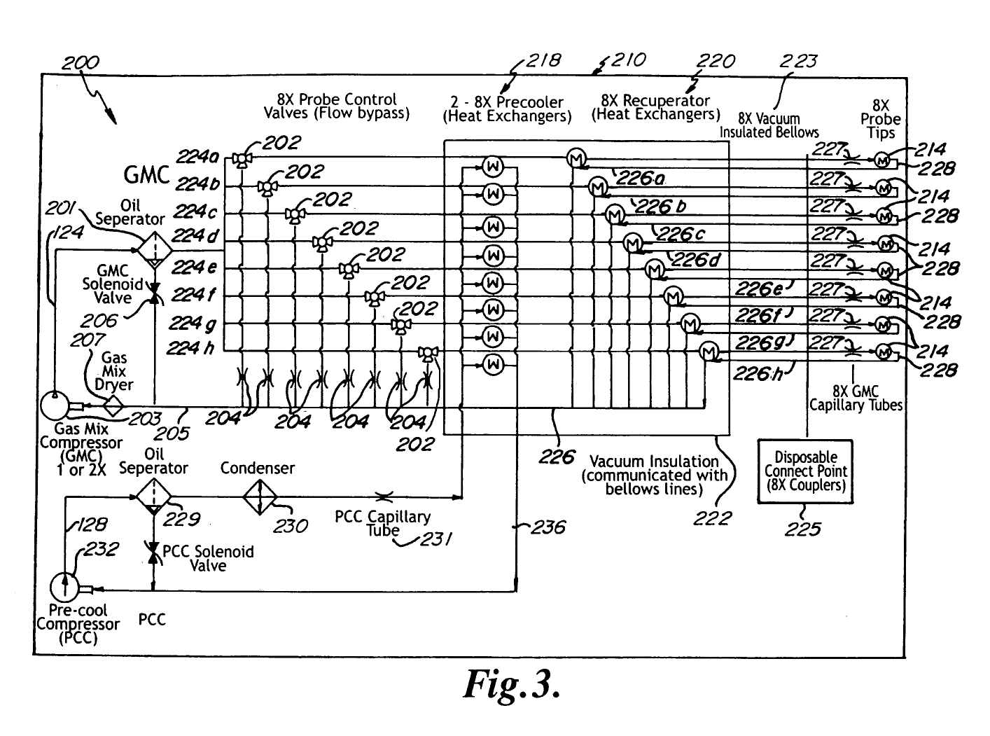

The present invention replaces the manifold system and the electric heater with a valve control system for independent thermal control of each probe. Referring now to FIG. 3, a cryosurgical system 200 for eight Joule-Thomson cryoprobes incorporating an individual control system is illustrated schematically. In general, high pressure primary refrigerant 124 is divided into a separate fluid path for each respective probe after passing through oil separator filter 201. In the embodiment illustrated in FIG. 3, eight separate refrigerant lines 224a-h are included. After primary refrigerant 124 is divided into refrigerant lines 224a-h, a probe control valve 202 is inserted into each line. The probe control valve 202 is a three way valve, preferably a three way solenoid valve, for selectively directing gas away from cryostat 210. Gas directed away from cryostat 210 is directed ultimately back to gas mix compressor 203. Valves 202 can each selectively allow all gas to pass through into the probes, reroute all gas back to the compressor 203, or allow a predetermined amount of gas to both the probes and the compressor 203. Return flow to compressor 203 of refrigerant lines 224a-h first passes through restrictor 204 in each respective line for mass flow balancing of the entire system 200. Restrictor 204 can be, for example, capillary tubing, orifices, or needle valves. Refrigerant lines 224a-h are then combined to a single refrigerant line 205. The combined refrigerant line 205 is in communication with oil separator filter 201 by way of adjustable solenoid valve 206 for pressure balancing. Refrigerant line 205 is directed through gas mix 207 before entering gas mix compressor 203. Refrigerant line 205 can also include a bypass flow heat rejecter for rejecting excess heat in the refrigerant returning to the compressor.

When flow bypass valves 202 are closed, refrigerant lines 224a-h enter the cryostat 210 and each line is cooled by high pressure secondary refrigerant 128. A secondary refrigerant line 128 flows through oil separator 229, then into condenser 230. Secondary refrigerant line 128 is expanded to a lower temperature through capillary 231 and then directed to the pre-cool heat exchanger 218. The resulting low pressure secondary refrigerant 236 then returns to the secondary compressor 232 to be repressurized. Since the secondary refrigerant 128 does not flow into the probes 214 (which are brought into direct contact with the patient), a higher pressure can be safely used for the secondary refrigerant 128, 230 than the primary refrigerant lines 124.

Cryostat heat exchanger module 210 may contain both a pre-cool heat exchanger, or pre-cooler 218, and a recuperative heat exchanger, or recuperator 220 for each refrigerant line 224a-h respectively. A vacuum insulated jacket 222 surrounds the cryostat 210 to prevent ambient air from warming the refrigerant within the cryostat 210 and to prevent the outer surface of the cryostat 210 from becoming excessively cold.

The high pressure primary refrigerant lines 224a-h direct primary refrigerant 124 into the recuperator 220 where it is further cooled by the low pressure primary refrigerant lines 226a-h returning from the probes 214. The low pressure primary refrigerant lines 226a-h are colder than the high pressure primary refrigerant lines 224a-h because a low pressure primary refrigerant has undergone Joules-Thompson expansion in the probes 214. Recuperator 220 is preferably incorporated into the cryostat 210. Alternatively, tubing coils inside each probe 214 may act as recuperative heat exchangers in order to reduce insulation requirements and return low pressure refrigerant to the console.

After leaving the recuperator 220, high pressure primary refrigerant 124 flows into the vacuum insulated bellows section 223. Instead of the typical manifold where refrigerant is distributed into multiple flexible probes, the present invention utilizes couplers 225 to provide for the connection of disposable probe ends for contamination protection and durability. In one presently contemplated embodiment, eight probes 214 are individually connected to the gas mix compressor 203, but one of skill in the art will recognize that greater or fewer probes may be used depending on the needs of a particular procedure. In each probe 214, high pressure primary refrigerant 124 flows into a Joule-Thompson expansion element 227, such as a valve, orifice, or other type of flow constriction, located near the tip of each probe 214, where the high pressure primary refrigerant 124 is expanded isenthalpically to a lower temperature. In one presently preferred embodiment, the Joule-Thompson expansion elements 227 are capillary tubes. A low pressure primary refrigerant 228 then cools a heat transfer element mounted in the wall of the probe 214, allowing the probe to form ice balls that freeze diseased tissue. The low pressure primary refrigerant 228 then follows low pressure primary refrigerant lines 226a-h and travels through the recuperator 220 (where it serves to further cool the high pressure primary refrigerant 124), flows past the precooler 218 and back to the primary compressor 203 in the console, where it is compressed back into high pressure primary refrigerant 124 so that the above process can be repeated. The present invention requires active control of the valves 204 to maintain mass flow through the system when one or more individual probes are turned off.

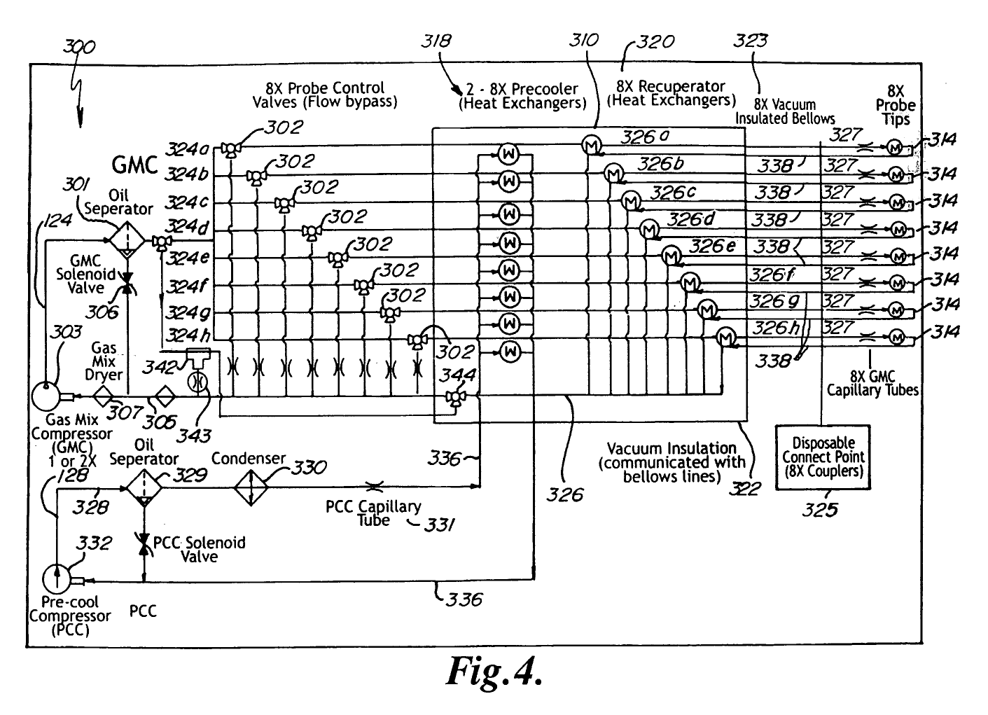

In an alternate embodiment, as illustrated in FIG. 4, the present invention includes a method to reverse the flow of the pressurized gas to avoid the heat exchangers so that hot gas can enter the probe for thawing the iceballs. The hot refrigerant gas flowing from the gas mix compressor is warm enough to heat the probes but it must be directed to the probes without flowing through the heat exchanger system.

As the heat cycle occurs after cooling, the system first must have the capability to individually cool each probe. Referring now to FIG. 4, a schematic view of a cryosurgical system 300 for eight Joule-Thomson cryoprobes 314 is illustrated incorporating an individual heating and cooling control system. In general, high pressure primary refrigerant 124 is divided into a separate fluid path for each respective probe after passing through oil separator filter 301. In the embodiment illustrated in FIG. 4, eight separate refrigerant fluid lines 324a-h are included. After primary refrigerant 124 is divided into refrigerant lines 324a-h, a probe control valve 302 is inserted into each line. The probe control valve 302 is a three way valve, preferably a three way solenoid valve, for selectively directing gas away from cryostat 310. Gas directed away from cryostat 310 is directed ultimately back to gas mix compressor 303. Return flow of high pressure primary refrigerant 124 first passes through restrictor 304 in each respective line for mass flow balance of the entire system 300. Refrigerant lines 324a-h are then combined to a single refrigerant line 305. The combined refrigerant line 305 is in communication with oil separator filter 301 by way of adjustable solenoid valve 306 for pressure balancing. Combined refrigerant line 305 is directed through gas mix dryer 307 before entering gas mix compressor 303.

When flow bypass valves 302 are closed, high pressure primary refrigerant 124 enters the cryostat 310 and each refrigerant line is cooled by high pressure secondary refrigerant 328. High pressure secondary refrigerant 328 flows through oil separator 329, and then through condenser 330 before it is expanded to a lower temperature through capillary 331. Secondary low pressure refrigerant 336 is then directed to pre-cool heat exchanger 318. The resulting low pressure secondary refrigerant 336 then returns to the secondary compressor 332 to be repressurized. Since the secondary refrigerant 328 does not flow into the probes 314 (which are brought into direct contact with the patient), a higher pressure can be safely used for the secondary refrigerant line 128 than the primary refrigerant lines 324a-h.

Cryostat heat exchanger module 310 may contain both a pre-cool heat exchanger, or pre-cooler 318, and a recuperative heat exchanger, or recuperator 320 for each refrigerant line 324a-h respectively. A vacuum insulated jacket 322 surrounds the cryostat 310 to prevent the ambient air from warming the refrigerant within the cryostat 310 and to prevent the outer surface of the cryostat 310 from becoming excessively cold.

The high pressure primary refrigerant 124 then continues into the recuperator 320 where it is further cooled by the low pressure primary refrigerant 338 returning from the probes 314. The low pressure primary refrigerant 338 is colder than the high pressure primary refrigerant 124 because it has undergone Joule-Thompson expansion in the plurality of probes 314. Recuperator 320 is preferably incorporated into the cryostat 310. Alternatively, tubing coils inside each probe 314 may act as recuperative heat exchangers in order to reduce insulation requirements and return low pressure refrigerant to the console.

After leaving the recuperator 320, high pressure primary refrigerant 124 flows into the vacuum insulated bellows section 323. Instead of the typical manifold where refrigerant is distributed into multiple flexible probes, the present invention utilizes couplers 325 to provide for the connection of disposable probe ends for contamination protection and durability. In one presently contemplated embodiment, eight probes 314 are individually connected to the gas mix compressor 303, but one of skill in the art will recognize that greater or fewer probes may be used depending on the needs of a particular procedure. In each probe 314, the high pressure primary refrigerant 124 flows into a Joule-Thompson expansion element 327, such as a valve, orifice, or other type of flow constriction located near the tip of each probe 314, where the high pressure primary refrigerant 124 is expanded isenthalpically to a lower temperature. In one presently preferred embodiment, the Joule-Thompson expansion elements are capillary tubes. The low pressure primary refrigerant 338 then cools a heat transfer element mounted in the wall of the probe 314, allowing the probe to form ice balls that freeze diseased tissue. The low pressure primary refrigerant 338 then follows low pressure primary refrigerant line 326a-h, travels through the recuperator 320 (where it serves to further cool the high pressure primary refrigerant 124), flows past the precooler 318 and back to the primary compressor 303 in the console, where it is compressed back into high pressure refrigerant 124 so that the above process can be repeated. The present invention requires active control of the valves 304 to maintain mass flow through the system when one or more individual probes 314 are turned off.

After the cooling cycle has begun, the high pressure primary refrigerant 124 can be used to rethaw the probes 314. High pressure primary refrigerant 124 passes through oil separator filter 301 before high pressure primary refrigerant 124 is divided into a separate fluid path for each respective probe 314. However, to warm the probes 314, high pressure primary refrigerant 124 flows into a three way control valve 340 that selectively directs high pressure primary refrigerant 124 to bypass the precooler 318 and recuperator stage 320 of cryostat 310. High pressure primary refrigerant 124 flows through two way valve 342 that is selectively in communication with pressure relief needle valve 343 that allows excess high pressure primary refrigerant 124 to flow back to gas mix compressor 303 under certain pressure conditions.

High pressure primary refrigerant 124 then continues into the heat exchanger 320 through three way diverter valve 344 from where high pressure primary refrigerant 124 is divided into flow refrigerant lines 326a-h and then directed to probes 314, respectively. The reverse flow scheme avoids the capillary tubes 327 before the probe tips. On the return flow, the refrigerant lines 326a-h can be directed back to the original return path at valve 302. It is envisioned that the reverse flow line could include a heater element for increasing the temperature of high pressure primary refrigerant 124. It is further envisioned that the lines could be insulated to decrease heat loss of high pressure primary refrigerant 124.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products.

1. A cryosurgical treatment system comprising:
a primary refrigerant circuit including a primary compressor for compressing a primary refrigerant, the primary refrigerant directed to a plurality of high pressure refrigerant lines, each high pressure primary refrigerant line including a three-way bypass valve for selectively diverting the high pressure primary refrigerant to a cryoprobe supply line or a compressor return line.

2. The cryosurgical treatment system of claim 1, further comprising:
a secondary refrigerant circuit including a secondary compressor for compressing a secondary refrigerant, the secondary refrigerant directed through a precooler for cooling high pressure primary refrigerant in the cryoprobe supply lines.

3. The cryosurgical treatment system of claim 2, further comprising an expansion element in the secondary refrigerant circuit to expand the secondary refrigerant prior to entering the precooler.

4. The cryosurgical treatment system of claim 2, further comprising a recuperator heat exchanger for cooling the high pressure primary refrigerant in the cryoprobe supply lines with a low pressure primary refrigerant returning from a plurality of cryoprobes.

5. The cryosurgical treatment system of claim 4, wherein each cryoprobe includes an expansion element to expand the high pressure primary refrigerant to form the low pressure primary refrigerant to cool a tip portion of the cryoprobe.

6. The cryosurgical treatment system of claim 4, wherein the precooler and the recuperator heat exchanger are insulated with a vacuum insulated jacket.

7. The cryosurgical treatment system of claim 1, wherein the three-way bypass valve comprises a three-way solenoid valve.

8. The cryosurgical treatment system of claim 1, wherein each compressor return line includes a mass flow restrictor.

9. The cryosurgical treatment system of claim 1, wherein the primary refrigerant circuit further comprises a three-way diverter valve between the primary compressor and the plurality of high pressure refrigerant lines, the three-way diverter valve selectively allowing the high pressure primary refrigerant to be directed to a low pressure side of a cryoprobe for heating a tip portion of the cryoprobe and wherein the high pressure primary refrigerant returns to the primary compressor through the three-way bypass valves and compressor return lines.

10. The cryosurgical system of claim 1, wherein each three-way bypass valve can divert a portion of the primary refrigerant through both the cryoprobe supply line and the compressor return line.

11. A method for selectively controlling temperatures of multiple cryoprobes during a cryosurgical procedure comprising:
providing a primary refrigeration circuit for pressurizing a high pressure primary refrigerant;
directing the high pressure primary refrigerant through a plurality of supply lines, each supply line including a bypass valve capable of selectively directing the high pressure primary refrigerant to a cryoprobe supply line or a compressor return line.

12. The method of claim 11, further comprising:
balancing a mass flow through the primary refrigeration circuit by positioning a mass flow restrictor in each compressor return line.

13. The method of claim 11, further comprising:
providing a secondary refrigeration circuit for pressurizing a secondary refrigerant;
cooling high pressure primary refrigerant in the cryoprobe supply lines with the secondary refrigerant in a precooler.

14. The method of claim 13, further comprising:
expanding the secondary refrigerant in an expansion element prior to cooling the high pressure primary refrigerant.

15. The method of claim 13, further comprising:
cooling the high pressure primary refrigerant in a recuperator heat exchanger with an expanded low pressure primary refrigerant returning from a cryoprobe.

16. The method of claim 16, further comprising:
expanding the high pressure primary refrigerant through an expansion element in the cryoprobe to form the expanded low pressure primary refrigerant.

17. The method of claim 11, further comprising:
diverting the high pressure primary refrigerant prior to the plurality of supply lines such that the high pressure primary refrigerant is directed to a low pressure side of a cryoprobe; and
heating a tip portion of the cryoprobe with the high pressure primary refrigerant to thaw tissue at a treatment site.
